(19) United States
(12) Patent Application Publication
(10) Pub. No.: US 2004/0147928 A1
(43) Pub. Date: Jul. 29, 2004

(54) Spinal stabilization system using flexible members

(76) Inventors: Michael E. Landry, Austin, TX (US); Larry T. Khoo, Studio City, CA (US)

Correspondence Address: MEYERTONS, HOOD, KIVLIN, KOWERT & GOETZEL, P.C., P.O. BOX 398, AUSTIN, TX, 78767-0398, US

(21) Appl. No.: 10/698,046

(22) Filed: Oct. 30, 2003

Related U.S. Application Data
(60) Provisional application No. 60/422,453, filed on Oct. 30, 2002.

(51) Int. Cl. A61F 2/30; A61B 17/56

(52) U.S. Cl. 606/61; 623/17.11

(57) ABSTRACT

A spinal stabilization system and method are provided for use in minimally invasive procedures. A plane of separated tissue may be established between adjacent vertebrae. In some embodiments, threaded members may be positioned in bone. Flexible members may be coupled to the threaded members. In an embodiment, flexible members may be used to position components of a spinal stabilization system proximate bone. Flexible members may maintain an alignment along a centerline of a threaded member. In some embodiments, a thickness of a flexible member may be varied to increase a stiffness of the flexible member.

### PRIORITY CLAIM

This application claims priority to U.S. Provisional Application No. 60/422,453 entitled “Spinal Stabilization System Using Flexible Members,” filed Oct. 30, 2002. The above-referenced provisional application is incorporated by reference as if fully set forth herein.

### BACKGROUND

1. Field of the Invention

The present invention generally relates to spinal stabilization systems. An embodiment of the invention relates to a system for use with minimally invasive surgical procedures. Spinal stabilization systems may include guides, threaded members, and/or coupling mechanisms.

2. Description of Related Art

Bone may be subject to degeneration caused by trauma, disease, and/or aging. Degeneration may destabilize bone and affect surrounding structures. For example, destabilization of a spine may result in alteration of a natural spacing between adjacent vertebrae. Alteration of a natural spacing between adjacent vertebrae may subject nerves that pass between vertebral bodies to additional pressure. Pressure applied to the nerves may cause pain and/or nerve damage. Maintaining the natural spacing between vertebrae may reduce pressure applied to nerves that pass between vertebral bodies. A vertebral stabilization procedure may be used to maintain the natural spacing between vertebrae and promote spinal stability.

Spinal stabilization may involve accessing a portion of the spine through soft tissue. Conventional stabilization systems may require a large incision and/or multiple incisions in the soft tissue to provide access to a portion of the spine to be stabilized. Conventional procedures may result in trauma to the soft tissue, for example, due to muscle stripping.

Spinal stabilization systems for a lumbar region of the spine may be inserted during a spinal stabilization procedure using a posterior spinal approach. Conventional systems and methods for posterolateral spinal fusion may involve dissecting and retracting soft tissue proximate the surgical site.

U.S. Pat. No. 6,530,929 to Justis et al. (hereinafter “Justis”), which is incorporated by reference as if fully set forth herein, describes minimally invasive techniques and instruments for stabilizing a bony structure in an animal subject. Justis provides a method for using an instrument to connect at least two bone anchors with a connecting element. The instrument is secured to the anchors and manipulated to place the connecting element in a position more proximate the anchors.

### SUMMARY

Spinal stabilization systems may include threaded members. The threaded members may be coupled to vertebrae. In some embodiments, threaded members may be coupled to pedicles. A threaded member may include a passage. In some embodiments, vertebrae to be stabilized may be accessed by a guide or flexible member inserted through a passage in a threaded member. A guide or flexible member may be coupled to a threaded member.

In a flexible member embodiment, stiffness of the flexible member may vary along a length of the flexible member. Stiffer sections of the flexible member may align a section of the flexible member through a centerline of a threaded member. In some embodiments, thickness of the flexible member may vary along a length of the flexible member.

Some spinal stabilization system embodiments may include coupling mechanisms. Coupling mechanisms may include, but are not limited to, connectors, threaded members, and elongated members. Connectors may engage threaded members positioned in adjacent vertebrae. An elongated member may be engaged by the connectors to couple the adjacent vertebrae. In some embodiments, a flexible member may be coupled to a passage through a threaded member.

Connectors may include rings to engage threaded members and/or locking mechanisms. Rings may include protrusions to engage threaded members. In some embodiments, rings may inhibit rotational movement of threaded members in bone during use. In a ring embodiment, the ring may be formed from a relatively soft material. In some embodiments, some surfaces of the ring may be treated to increase surface hardness.

A method for coupling adjacent vertebrae using a minimally invasive procedure may include positioning threaded members in vertebrae. In some embodiments, a flexible member may be coupled to a threaded member. In some embodiments, the method may include moving a separating member through soft tissue. The separating member may be moved from a position proximate a first vertebra to a position proximate a second vertebra. The separating member may separate the soft tissue on a plane between the first vertebra and the second vertebra such that damage to the soft tissue is reduced as compared with cutting the soft tissue. A coupling mechanism may be positioned in an opening at the surface of the body. The coupling mechanism may be moved through the plane of separated tissue to a position proximate the vertebrae. In some embodiments, flexible members may be used to guide the coupling mechanism into position proximate the vertebrae. The coupling mechanism may be coupled to threaded members positioned in the vertebrae.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a side view of an embodiment of a flexible member for a minimally invasive spinal stabilization system.

FIG. 2 depicts a side view of an embodiment of a flexible member for a minimally invasive spinal stabilization system.

FIG. 3 depicts a schematic of flexible members positioned in threaded members coupled to vertebrae.

FIG. 4 depicts a perspective view of an embodiment of a threaded member.

FIG. 5 depicts a cross-sectional representation of an embodiment of a threaded member.

FIG. 6 depicts a cross-sectional representation of an embodiment of a threaded member coupled to a driver and a flexible member.

FIG. 7 depicts a perspective view of an embodiment of a spinal stabilization system.

FIG. 8 depicts a perspective view of an embodiment of a spinal stabilization system for two vertebral levels.

FIG. 9 depicts a perspective view of an embodiment of a spinal stabilization system.

FIG. 10 depicts a top view of an embodiment of a spinal stabilization system.

FIG. 11 depicts a front view of an embodiment of a spinal stabilization system.

FIG. 12 depicts a cross-sectional representation of an embodiment of a spinal stabilization system.

FIG. 13 depicts a cross-sectional representation of an embodiment of a spinal stabilization system.

FIG. 14 depicts a cross-sectional representation of an embodiment of a spinal stabilization system.

FIG. 15 depicts a perspective view of an embodiment of a ring for a spinal stabilization system.

FIG. 16 depicts a perspective view of an embodiment of a ring for a spinal stabilization system.

FIG. 17A-FIG. 17E depict schematic views of a method of preparing a vertebra for a minimally invasive stabilization procedure.

FIG. 18A-FIG. 18D depict schematic views of a method of preparing a vertebra for a minimally invasive stabilization procedure.

FIG. 19 depicts a perspective view of a c-shaped dilator positioned proximate a pedicle.

FIG. 20A-FIG. 20C depict schematic views of a method of preparing a vertebra for a minimally invasive stabilization procedure.

FIG. 21A and FIG. 21B depict front views of an embodiment of a threaded member being coupled to an embodiment of a driver.

FIG. 22A-FIG. 22E depict schematic views of a method for coupling a threaded member to a first vertebra.

FIG. 23A-FIG. 23D depict schematic views of a method for coupling a threaded member to a second vertebra.

FIG. 24A and FIG. 24B depict schematic views of an embodiment of an estimator tool determining a length of a rod.

FIG. 25A-FIG. 25D depict perspective views of an embodiment of a coupling mechanism.

FIG. 26A-FIG. 26E depict schematic views of a method for coupling an embodiment of a coupling mechanism to vertebrae.

FIG. 27A-FIG. 27C depict schematic views of a method for coupling an embodiment of a coupling mechanism to vertebrae.

While the invention is susceptible to various modifications and alternative forms, specific embodiments thereof are shown by way of example in the drawings and will be described in detail. The drawings may not be to scale. It should be understood, however, that the drawings and detailed description are not intended to limit the invention to the particular form disclosed, but on the contrary, the intention is to cover all modifications, equivalents, and alternatives falling within the spirit and scope of the present invention as defined by the appended claims.

### DETAILED DESCRIPTION

A spinal stabilization system may be implanted using a minimally invasive procedure to reduce trauma to surrounding soft tissue. Spinal stabilization systems may include guides, coupling mechanisms, and threaded members. Minimally invasive procedures may provide limited visibility in vivo. Positioning a spinal stabilization system using a minimally invasive procedure may include using guides to position a coupling mechanism and/or threaded members in bone.

Minimally invasive procedures may reduce trauma to soft tissue surrounding a surgical site (e.g., retraction and/or severing of muscle tissue proximate the surgical site may be reduced). In addition, minimizing an area required to access a portion of the spine may reduce exposure of the spine. Recovery time for surgical stabilization procedures may be reduced when a minimally invasive procedure is used.

Components of spinal stabilization systems may include materials such as, but not limited to, stainless steel, titanium, titanium alloys, ceramics, and/or polymers. Some components of the spinal stabilization system may be autoclaved and/or chemically sterilized. Components that may not be autoclaved and/or chemically sterilized may be made of sterile materials. Components made of sterile materials may be placed in working relation to other sterile components during assembly of a spinal stabilization system.

Spinal stabilization systems may be used to correct problems in lumbar, thoracic, and/or cervical portions of a spine resulting from injury and/or disease. Various embodiments of a spinal stabilization system may be used from the C1 vertebra to the sacrum. For example, a spinal stabilization system may be implanted in a lumbar portion of a spine using a posterior approach. In some embodiments, spinal stabilization systems may be implanted using a lateral approach or an anterior approach.

In some cases, spinal stabilization systems may be implanted bilaterally (i.e., on opposite sides of a spine). Alternatively, spinal stabilization systems may be used unilaterally (i.e., on a single side of a spine). For example, a spinal stabilization system used in a thoracic region may be used on a single side of a spine.

In some embodiments, a spinal stabilization system may stabilize a vertebral level. A vertebral level may include two adjacent vertebrae and an intervertebral disc between the vertebrae. In some embodiments, a spinal stabilization system may stabilize two or more vertebral levels.

In some embodiments a spinal stabilization system may be inserted into a patient using a minimally invasive procedure. After installation of the spinal stabilization system, interbody work may be performed. Interbody work may be work performed on an intervertebral disc. For example, a discectomy may be performed and a fusion device may be positioned in the formed disc space. After the interbody work is completed, a final position of the spinal stabilization system may be set.

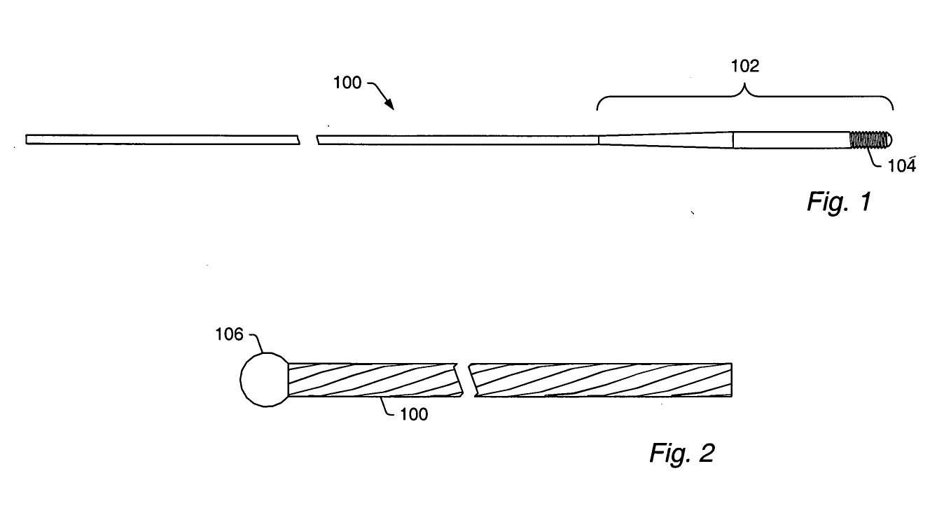

Guides may be used during minimally invasive procedures to place components of spinal stabilization systems proximate vertebrae. Embodiments of guides are depicted in FIG. 1 and FIG. 2. Guides may include, but are not limited to wires, cables, dilators, flexible members, rigid members, and/or conduits. In some embodiments, a guide may be coupled to a portion of bone to be stabilized. In certain instances, a guide may be coupled to a threaded member after implantation of the threaded member into bone. In alternative embodiments, a guide may be coupled to a threaded member prior to implantation of the threaded member into bone.

FIG. 1 depicts flexible member 100 for use as a guide. Flexible member 100 may be formed from titanium, stainless steel, synthetic materials (e.g., nylon), and/or shape memory alloys (e.g., titanium alloys such as nitinol). Flexible members may have lengths greater than about 10 cm. In some embodiments, flexible members may have lengths greater than about 20 cm. In some embodiments, flexible members may have lengths greater than about 30 cm.

Stiffness of flexible member 100 may vary along a length of the flexible member. In some embodiments, stiffer sections of flexible member 100, such as engagement section 102, may allow for small alignment variability proximate a threaded member. For example, engagement section 102 may have a stiffness sufficient to allow flexible member 100 to maintain alignment along a centerline of a threaded member within about 0.6 cm to about 3.2 cm of a threaded member head. In an embodiment, the engagement section may have a stiffness sufficient to allow the flexible member to maintain alignment along a centerline of a threaded member within about 1.3 cm of a threaded member head. Engagement section stiffness may affect alignment of components of a spinal stabilization system proximate a surgical site.

In some embodiments, stiffness of flexible member 100 may vary along a length of the flexible member. In certain embodiments, a thickness of flexible member 100 may vary along a length of the flexible member. In an example, an end portion of the flexible member may be stainless steel and relatively inflexible, while a majority of the flexible member is formed of stranded wire that is flexible. Alternatively, different materials may be used to form sections of flexible member 100. As shown in FIG. 1, engagement section 102 may be thicker than other portions of flexible member 100. Thus, engagement section 102 may be stiffer than other sections of flexible member 100.

In some embodiments, engagement section 102 may couple to a threaded member and/or a portion of bone. Engagement section 102 may include threading 104. Threading 104 may engage a portion of a threaded member and/or bone. Engagement section embodiments may include various surface configurations to couple flexible member 100 to a threaded member and/or bone. For example, engagement section 102 may include, but is not limited to, hex sections, hexalobular sections, tapered sections, beads, knots, keyed openings, coatings, roughened surfaces, and/or threading.

FIG. 2 depicts an embodiment of a flexible member. Flexible member 100 may include stop 106 (e.g., a bead or a knot). A diameter of stop 106 may be greater than a diameter of a passage through a portion of a threaded member, fastener, setscrew, or other member through which flexible member 100 passes.

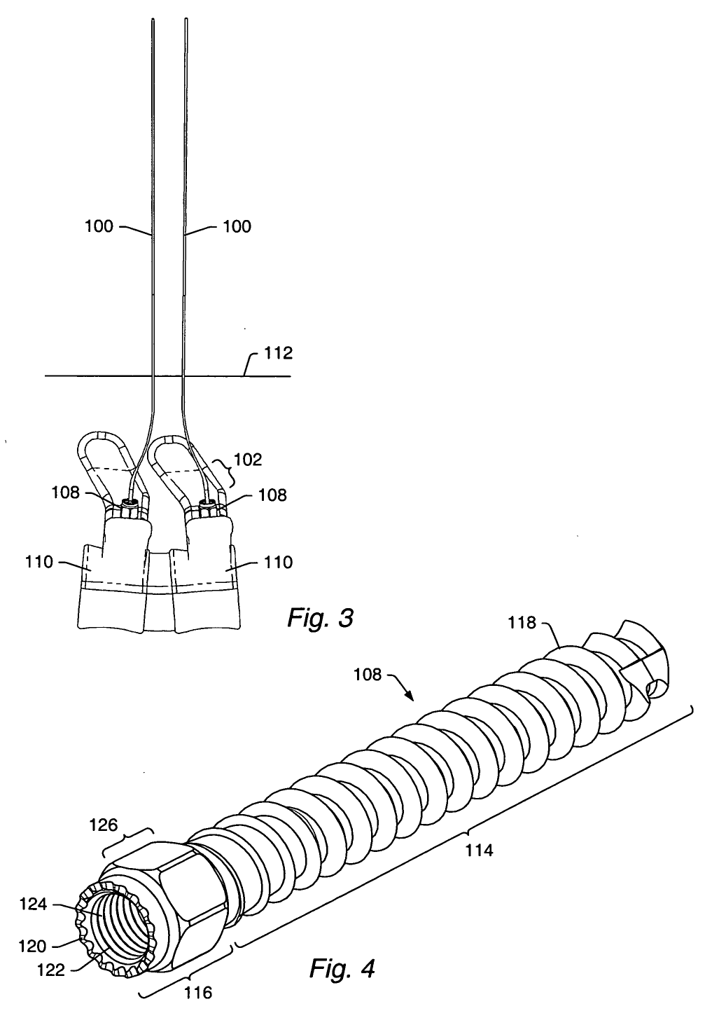

FIG. 3 depicts threaded members 108 positioned in vertebrae 110. Threaded members 108 may couple flexible members 100 to vertebrae 110. Flexible members 100 may exit at body surface 112 through an opening in soft tissue. In some embodiments, a soft tissue opening may have a length less than a distance between vertebrae that are to be stabilized. The elastic nature of the skin and tissue may allow movement of tissue without the need to form an incision that spans or is greater than the full length of the spinal stabilization system to be installed in a patient. In some embodiments of single vertebral level stabilization systems, an incision formed in the skin may be less than about 4 cm in length. In some embodiments, an incision formed in the skin may be less than about 3 cm in length. In some embodiments, an incision formed in the skin may be less than about 2.5 cm in length.

In some embodiments, a flexible member that is coupled to a vertebra may be used to adjust a position of threaded members 108 and of a vertebra that the threaded member is coupled to. For example, a vertebra may slip and/or be out of alignment with adjacent vertebrae due to injury and/or disease. A flexible member attached to the misaligned vertebra may be maneuvered from above body surface 112 to adjust alignment of the vertebra. Flexible members 100 may be maneuvered manually with or without the aid of a mechanical device. Realigning the vertebrae may be referred to as reduction. Reduction may be used in conjunction with multi-level spinal stabilization systems.

Threaded members 108 may include any elongated member securable in bone. A threaded member may be, but is not limited to, a screw, a barb, a nail, a brad, or a trocar. An instrumentation set may provide threaded members in various lengths to accommodate variability in vertebral bodies. The threaded members may be color coded and/or stamped with indicia indicating lengths of the threaded members. For example, threaded members may be provided in 12 mm, 13 mm and 14 mm lengths. The lengths of the threaded members may be stamped on a side of the threaded member head. The 12 mm threaded members may have a gold color, the 13 mm threaded members may have a green color, and the 14 mm threaded members may have a magenta color. If desired, other colors may be used.

Each threaded member provided in an instrumentation set may have substantially the same thread profile. In an embodiment, the thread may have about a 4 mm major diameter and about a 2.5 mm minor diameter with a cancellous thread profile. Threaded members with other thread dimensions and/or thread profiles may also be used. A thread profile of the threaded members may allow for maximizing bone purchase.

Rescue threaded members may also be provided in an instrumentation set. A rescue threaded member may be positioned in a previously deformed threaded member opening in a vertebra. The rescue thread may have the same thread pitch as the regular threaded members. The rescue threaded members may have a larger thread major diameter and the same thread minor diameter as the regular threaded members. For example, if a regular threaded member has about a 4 mm major thread diameter and about a 2.5 mm minor thread diameter, a corresponding rescue threaded member may have about a 4.5 mm major thread diameter thread and about a 2.5 mm minor thread diameter. Rescue threaded members may be separated from regular threaded members in an instrumentation set. Rescue threaded members may be a different color than regular threaded members. For example, rescue thread members may be blue. Different shades of the color used for the rescue threaded members may be used to distinguish rescue threaded members of different lengths.

A threaded member embodiment is depicted in FIG. 4. Threaded member 108 may include shank 114 and head 116. In some embodiments, shank 114 may include threading 118 to engage vertebral bone. In some embodiments, threading 118 may include self-tapping starts to facilitate insertion into bone. In some embodiments, head 116 of threaded member 108 may include protrusions 120.

Head 116 may include passage 122 to allow threaded member 108 to couple to tools, locking mechanisms, and/or coupling mechanisms. In some embodiments, passage 122 may include threading 124. Threading 124 may be used to engage a locking mechanism.

Threaded member 108 may include various surface configurations to engage tools (e.g., drivers), coupling mechanisms, rings, and/or locking mechanisms (e.g., setscrews and/or lock nuts). For example, threaded member 108 may include, but is not limited to including, hex sections, hexalobular sections, tapered sections, beads, knots, keyed openings, coatings, roughened surfaces, and/or threading. In some embodiments, threaded member 108 includes tool section 126 to couple to a driving tool during insertion.

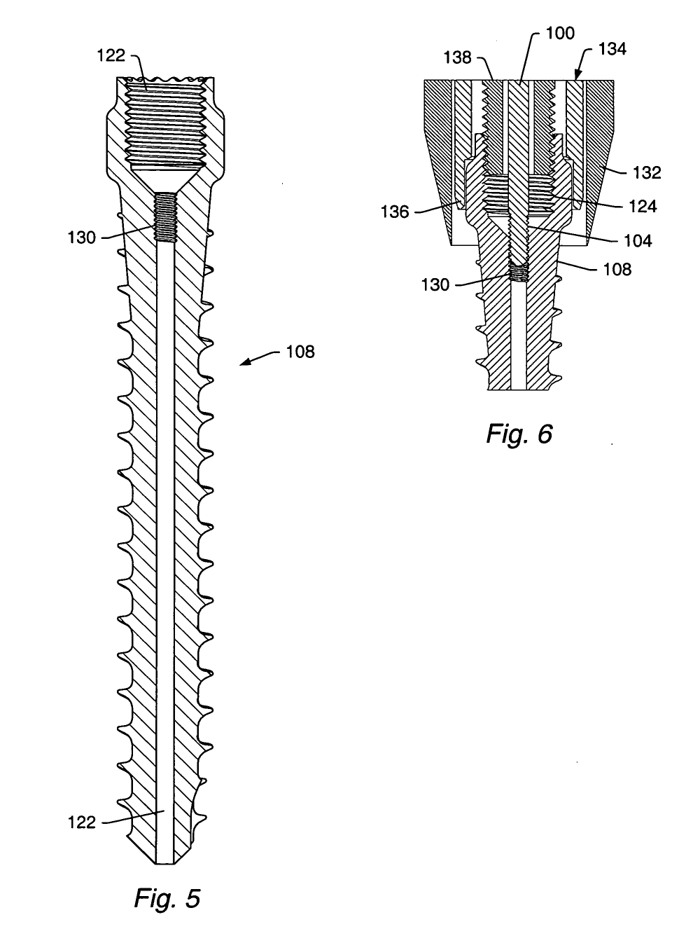

FIG. 5 depicts a cross-sectional view of an embodiment of threaded member 108. Passage 122 may extend through the head and the shank of threaded member 108. In some embodiments, a guide may be placed in passage 122 to allow threaded member 108 to be positioned at a desired location. A diameter of passage 122 may vary along a length of threaded member 108. Section 130 may be configured to engage a guide (e.g., a flexible member). Section 130 may include threading and/or another engagement mechanism to engage the guide.

FIG. 6 depicts a cross-sectional view of threaded member 108 positioned in dilator 132 and coupled to threaded member driver 134. Dilator 132 may enlarge an opening in soft tissue for insertion of tools and/or components of a spinal stabilization system. Outer conduit 136 of threaded member driver 134 engages an outer surface of threaded member 108. Inner conduit 138 of threaded member driver 134 may engage threading 124 of threaded member 108. Connecting inner conduit 138 to threaded member 108 may inhibit unintentional release of the threaded member from driver 134. Threading 104 of flexible member 100 may engage section 130 of threaded member 108.

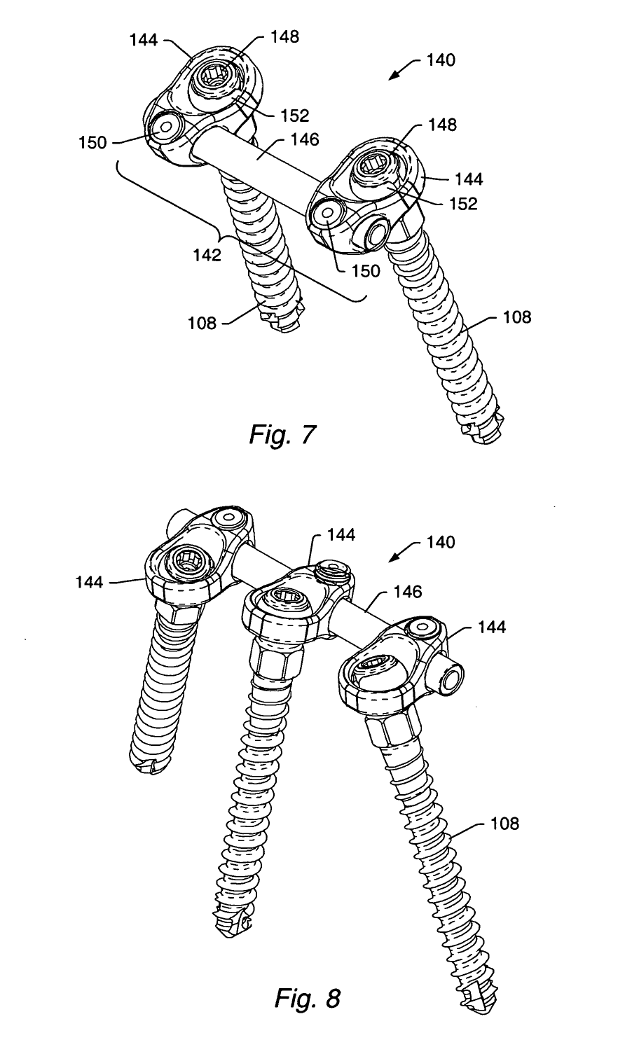

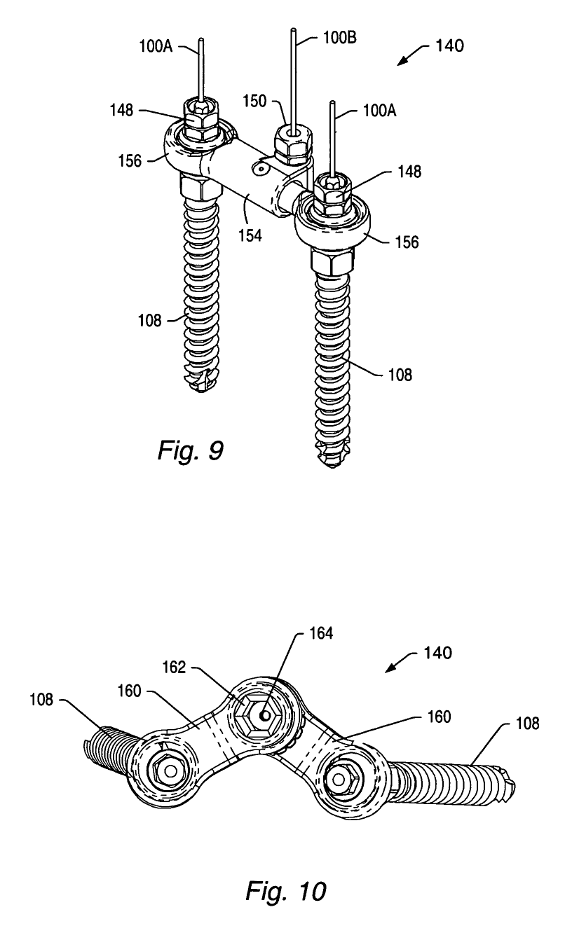

FIGS. 7-11 depict embodiments of spinal stabilization systems that may be formed using a minimally invasive surgical procedure. In some embodiments, spinal stabilization systems 140 may be used to provide stability to one or more vertebral levels. FIGS. 7 and 9 depict embodiments of spinal stabilization systems that may be used to stabilize a single vertebral level. A single vertebral level includes a first vertebra and a second vertebra adjacent to the first vertebra. FIG. 8 depicts an embodiment of a spinal stabilization system that may be used to stabilize two vertebral levels.

FIG. 7 depicts spinal stabilization system 140 having coupling mechanism 142 and threaded members 108. Coupling mechanisms may include, but are not limited to including, plates, elongated members (e.g., links, rods and dumbbell shaped members), connectors, or combinations thereof. Coupling mechanism 142 may include connectors 144, elongated member 146, locking mechanisms 148, setscrews 150, and/or rings 152. Connectors 144 may couple threaded members 108 to elongated member 146 to stabilize one or more vertebral levels. Locking mechanisms 148 and/or rings 152 may engage a portion of threaded member 108 to couple the threaded member to connector 144.

Coupling mechanisms 142 used in spinal stabilization systems may be adjustable. As shown in FIGS. 7 and 8, connectors 144 may be positioned along elongated member 146 to allow for a coupling mechanism of varying length. A length of coupling mechanism 142 may be fixed during manufacture, prior to surgery, or after insertion in the body.

As shown in FIG. 9, a coupling mechanism embodiment may include adjustable member 154 having coupling sections 156. After coupling sections 156 are coupled to threaded members 108 positioned in vertebrae, setscrew 150 may be advanced to inhibit movement of the coupling sections relative to each other. Portions of locking mechanisms 148 and a portion of setscrew 150 may be sheared off to allow for removal of flexible members 100A, 100B.

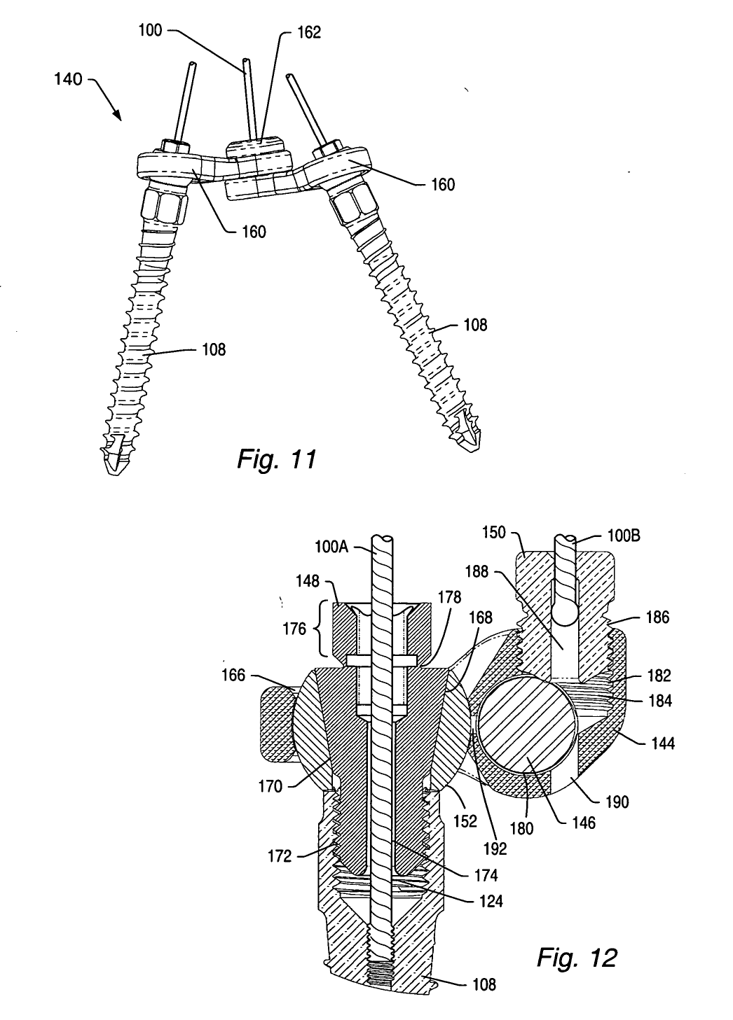

FIG. 10 and FIG. 11 depict embodiments of single level spinal stabilization systems 140. Spinal stabilization systems 140 may include links 160. Position of links 160 relative to each other may be set by tightening limiter 162. In some embodiments, limiter 162 may include threaded opening 164. A flexible member with a threaded ended may be coupled to threaded opening 164. FIG. 11 depicts limiter 162 with flexible member 100 extending from the limiter. A driver may be advanced down flexible member 100 to position a drive head in limiter 162. The driver may be rotated to allow limiter 162 to be tightened or loosened.

During a spinal stabilization procedure, links 160 may advantageously be positioned out of the way during interbody work. In some embodiments, links 160 may be originally positioned to provide some distraction to vertebrae that threaded members 108 are coupled to. A fusion procedure may be performed through the incision used to insert spinal stabilization system 140 in the patient. After the fusion procedure, position of links 160 relative to each other may be adjusted to provide compression to an installed fusion device. A driver may be advanced down flexible member 100. The driver may be used to tighten limiter 162 so that the position of links 160 are set relative to each other. After limiter 162 is tightened, the driver and flexible member 100 may be removed from spinal stabilization system 140.

FIG. 12 depicts a cross-sectional view of a spinal stabilization system. An opening in connector 144 includes inner surface 166. Inner surface 166 may engage a portion of a ring, a threaded member, and/or a locking mechanism. In some embodiments, inner surface 166 of the opening may be shaped to correspond to a contour of a portion of ring 152.

Inner surface 166 may be surface treated or include a liner, coating, and/or covering. Surface treatment (e.g., texturing and/or roughening), liners, coatings, and/or coverings may be used to adjust frictional and/or wear properties of material defining the opening. Texturing inner surface 166 may increase a coefficient of friction between connector 144 and ring 152. In some embodiments, an outer surface of ring 152 may be textured. In certain embodiments, inner surface 166 and an outer surface of ring 152 that engages inner surface 166 may both be textured to increase a coefficient of friction between connector 144 and the ring.

In general, any treatment that transforms a relatively smooth surface into a roughened surface having an increased coefficient of friction may be used to treat inner surface 166 and/or an outer surface of ring 152. Methods for forming a roughened surface include, but are not limited to sanding, forming grooves within a surface, ball peening processes, electric discharge processes, and/or embedding hard particles in a surface.

In some embodiments, ring 152 and locking mechanism 148 may be used to couple threaded member 108 to connector 144. Ring 152 may include, but is not limited to, a swivel and/or one or more crescents. A shape of an outer surface of ring 152 may allow polyaxial motion of the ring prior to expansion of the ring against connector 144. Polyaxial motion of ring 152 may allow connector 144 to be oriented in a desired position relative to vertebrae regardless of the insertion angle of threaded member 108 in a vertebra.

In some ring embodiments, different sections of the ring may have varying hardness. Hardness of sections of ring 152 may be varied by using methods including, but not limited to using materials varying in hardness for different sections of ring 152, utilizing surface treatment, and/or combinations thereof. Surface treatment to increase a hardness of a surface may include, but is not limited to, coating or treating a surface to produce a hardened layer (e.g., a titanium nitride layer), anodizing a surface, and/or implanting iron into the ring.

In some embodiments, outer surface of ring 152 may be formed of a relatively soft material as compared to the material used to form inner surface 166 of connector 144. For example, ring 152 may be formed from a soft biocompatible metal (e.g., substantially pure titanium). Utilizing a soft material may increase an ability of texturing and/or roughening of inner surface 166 of connector 144 to deform ring 152 and/or to frictionally lock with the ring. As locking mechanism 148 is advanced through ring 152, locking mechanism tapered section 168 may engage ring tapered section 170, causing ring 152 to expand outwards. Ring tapered section 170 may include a surface treatment to reduce gall stress between ring 152 and locking mechanism 148. Gall stress may be reduced by treating ring tapered section 170 with a surface treatment to increases a hardness and/or a smoothness of the ring tapered section.

Locking mechanisms may include several sections to engage different components of a spinal stabilization system. Threading 172 on locking mechanism 148 may be used to engage threading 124 in a passage of threaded member 108. A locking mechanism embodiment may include passage 174 through locking mechanism 148. In some embodiments, passage 174 in locking mechanism 148 may align with a passage of threaded member 108. Flexible member 100A coupled to threaded member 108 using threading 104 may pass through passage 174.

Locking mechanism 148 may include tool portion 176. Tool portion 176 may include various configurations (e.g., threading, hexalobular connections, hexes) for engaging a tool (e.g., a driver). Locking mechanism 148 may include groove 178. Groove 178 may allow tool portion 176 of locking mechanism 148 to shear off after the locking mechanism has been tightened and/or advanced to a pre-determined depth. In some embodiments, a wall thickness of locking mechanism 148 may be thinner proximate groove 178.

Elongated member 146 may be coupled to one or more connectors to stabilize adjacent vertebrae. Elongated member 146 may be positioned in opening 180 of connector 144. Setscrew 150 may be advanced in setscrew opening 182 to engage a portion of elongated member 146. Setscrew 150 may inhibit movement of elongated member 146. Setscrew opening 182 may include threading 184 to engage threading 186 on setscrew 150.

Setscrew 150 may include passage 188 to couple to a guide (e.g., a flexible member). Passage 188 may vary in diameter. In some embodiments, flexible member 100B may be positioned in passage 188 to aid in locating a position of setscrew 150. By varying the diameter of passage 188, a stop of the flexible member (as depicted in FIG. 2) may inhibit removal of the flexible member from setscrew 150. Passage 188 of setscrew 150 may align with passage 190 of connector 144 to allow a flexible member 100B to be positioned in setscrew 150 after the setscrew is coupled to connector 144 and before elongated member 146 is positioned in opening 180 of connector 144.

In some embodiments, material between an opening in connector 144 for ring 152 and opening 180 may be removed for ease of manufacturing to form cut-out 192. In some embodiments, cut-out 192 may reduce an area of inner surface 166 that contacts ring 152.

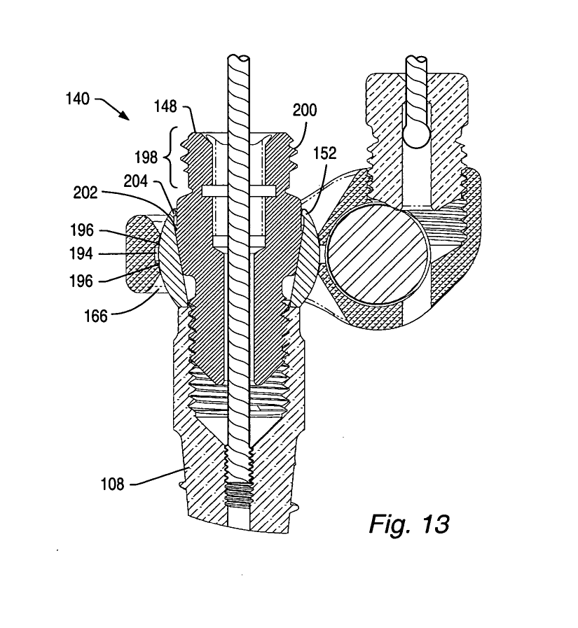

FIG. 13 depicts an embodiment of spinal stabilization system 140. Inner surface 166 may have recessed portion 194. Recessed portion 194 decreases a surface area of ring 152 contacting wall 166. In some embodiments, decreasing a contact area may increase pressure at contact points 196 as the locking mechanism is advanced. Pressure applied at points 196 may deform ring 152 against a wall of the connector. Thus, movement of the ring (e.g., rotational and/or axial) in the opening may be inhibited when locking mechanism 148 is fully inserted in threaded member 108.

Tool section 198 of locking mechanism 148 may include threading 200. Threading 200 may engage a tool. For example, a driver may couple to tool section 198 to advance locking mechanism 148.

In some embodiments, selected surfaces of locking mechanism 148 may be formed to engage ring 152. For example, locking mechanism 148 may include ledge 202 to engage finger 204 on ring 152 to inhibit removal of locking mechanism 148 from ring 152.

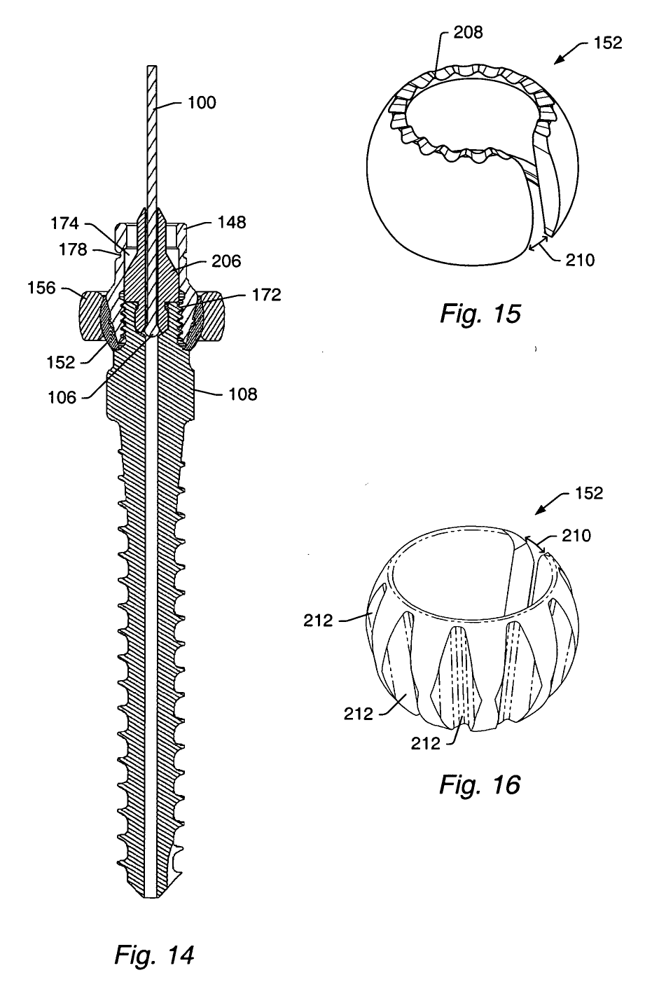

FIG. 14 depicts a cross-sectional view of an embodiment of coupling section 156 of the spinal stabilization system embodiment depicted in FIG. 9. In some embodiments, locking mechanism 148 may include a guide stop. Locking mechanism 148 may be positioned between ring 152 and threaded member 108. Locking mechanism 148 may include threading 172 to engage threaded member 108. In some embodiments, flexible member 100 may be coupled to threaded member 108 using guide stop 206. Stop 106 may have a diameter greater than a diameter of guide stop 206 to inhibit removal of flexible member 100 from threaded member 108. Passage 174 may have a variable diameter that inhibits removal of guide stop 206 from locking mechanism 148. A portion of locking mechanism 148 may be sheared off at groove 178. In an embodiment, guide stop 206 and flexible member 100 may be removed after a portion of locking mechanism 148 has been sheared off.

FIGS. 15 and 16 depict embodiments of rings 152 that may be used in combination with connector 144. FIG. 15 is a perspective view of ring 152 emphasizing a bottom surface of the ring. Ring 152 may include protrusions 208 on a lower surface to engage protrusions 120 on threaded member 108 (shown in FIG. 4). Engagement of ring protrusions 208 and threaded member protrusions 120 may inhibit rotational movement of a threaded member after ring 152 has expanded. Ring 152 may also include gap 210 to increase flexibility of the ring. Increased flexibility of ring 152 may be desired to allow for expansion of the ring as a locking mechanism is advanced and/or to allow for compression of the ring. Ring 152 may be compressed to allow for insertion of the ring into a connector.

As shown in FIG. 16, ring 152 may include indentations 212. Indentations 212 may increase flexibility of ring 152. In addition, indentations 212 may reduce a surface area on an outer surface of ring 152 that contacts an inner surface of a connector. Reducing the surface area of ring 152 contacting the wall of the connector may increase pressure at contact points between ring 152 and an inner surface of the connector. Increasing pressure at contact points may increase an ability of ring 152 to frictionally lock with the wall. In some embodiments, ring 152 may be pre-positioned in the connector during manufacturing. Alternatively, the ring may be positioned in the connector prior to insertion into a patient.

Minimally invasive procedures may include locating a surgical site and a position for an opening in the body to access the surgical site. In some spinal stabilization system insertion procedures, an incision may be made through the skin of a patient at a location between vertebrae that are to be stabilized. The skin incision may be a relatively small opening. In some embodiments, the skin opening may be less than 4 cm. In some embodiments, the skin opening may be less than 3 cm. In some embodiments, the skin opening may be less than 2.5 cm. The elasticity of skin and tissue may allow the incision and tissue to be moved to desired locations so that the skin incision does not have to be lengthened during a spinal stabilization system insertion procedure.

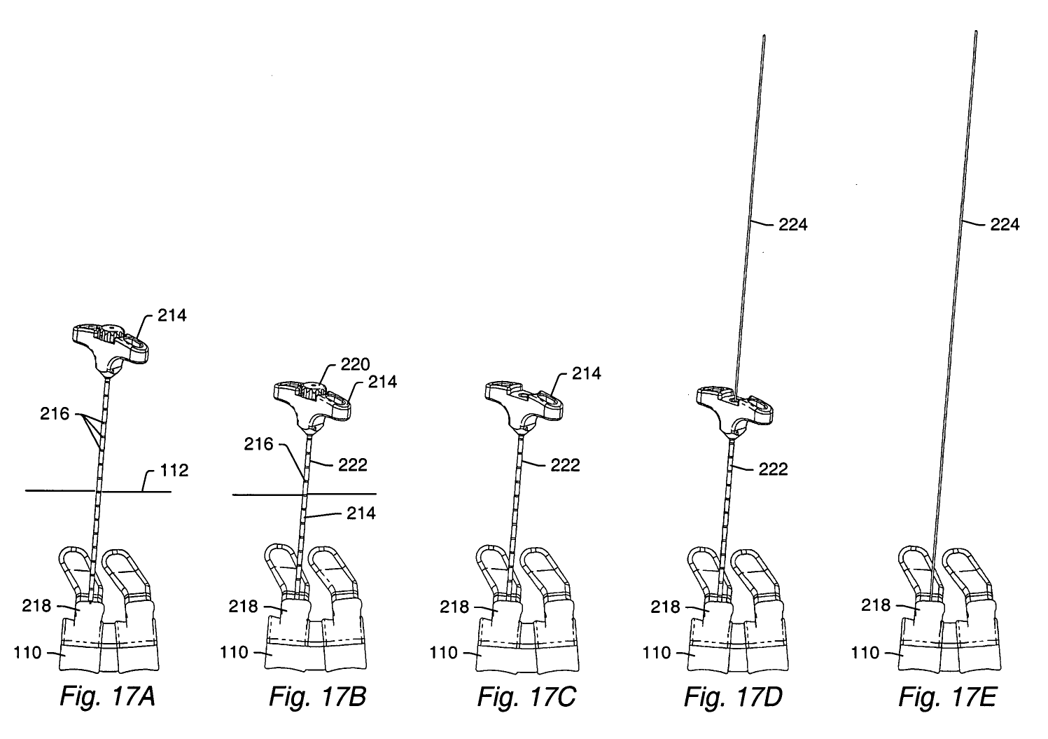

Fluoroscopic images may be used to determine a location for an initial incision. After the initial incision is made, a separating member may be inserted into the incision and advanced through soft tissue to a vertebra. FIG. 17A depicts separating member 214 positioned adjacent to vertebra 110. In some embodiments, separating member 214 may be a biopsy needle (e.g., a Jamshidi® biopsy needle). A fluoroscope may be used to confirm the position of separating member 214 relative to vertebra 110. Fluoroscopic images may be used to determine an insertion path for the separating member through a pedicle and into a vertebral body. Separating member 214 may include indicia 216. When a tip of separating member 214 is positioned on pedicle 218, a first measurement may be noted using indicia 216.

FIG. 17B depicts a position of separating member 214 after the separating member has been advanced into pedicle 218 of vertebra 110. In some procedures, the separating member may be advanced using a mallet. In some embodiments, a fluoroscope may be used to monitor the position of separating member 214 as the separating member is advanced. After separating member 214 has been advanced to a pre-determined depth, a second measurement may be noted using indicia 216. An approximate length of a threaded member may be determined by taking the difference between the two measurements.

Separating member 214 may include pointed member 220 and shaft 222. In some embodiments, after separating member 214 has been positioned in pedicle 218, pointed member 220 may be removed from shaft 222. FIG. 17C depicts separating member 214 after the pointed member has been removed from shaft 222.

FIG. 17D depicts rigid member 224 positioned through shaft 222 in an opening in pedicle 218. After rigid member 224 is positioned in the pedicle opening, shaft 222 of separating member 214 may be removed from the body. FIG. 17E depicts rigid member 224 after removal of the shaft.

A rigid member may have sufficient length to allow a surgeon or member of a surgical team to maintain a hold on the rigid member at all times. When the rigid member is being inserted through a passage in an instrument, the rigid member may be held near a dilator and/or near an incision in the skin. When the instrument is positioned in the patient, the rigid member may be held near a proximal end of the rigid member. Maintaining constant contact with the rigid member may inhibit removal of the rigid member and/or undesired advancement of the rigid member into the vertebra. In some embodiments, the rigid member may be K-wire that has length over about 25 cm. In some embodiments, the rigid member may have a length of about 45 cm. In some embodiments, a distal end of the rigid member may have a blunt tip. In some embodiments, a distal end of the rigid member may have a sharp or pointed tip.

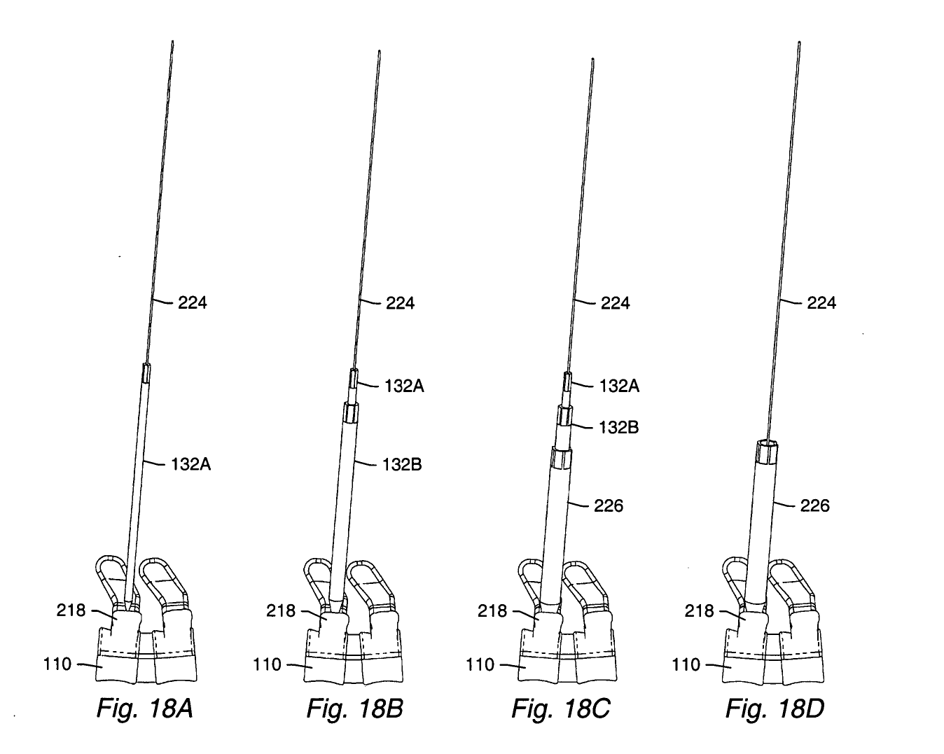

A dilator may be moved down a rigid member placed in a pedicle. FIG. 18A shows dilator 132A placed over rigid member 224 and against pedicle 218. Larger dilators may be placed over smaller dilators to form a working space that allows for the insertion of instruments and/or a threaded member of a spinal stabilization system. FIG. 18B and FIG. 18C depict small dilator 132A with larger dilators that expand the working space. The dilators may be rotated during insertion to facilitate separation of tissue. Dilator 132B, and dilator 226 of increasing diameter relative to small dilator 132A may be positioned in an opening. Three, four, five or more sequentially sized dilators may be used to form a working space. A largest dilator that is used may have an open channel down a side of the dilator. The channel may allow for instruments, such as a separating member, to be moved from a first vertebra to a second vertebra. Smaller dilators may be removed after insertion of a largest dilator. FIG. 18D depicts dilator 226 after removal of the smaller dilators.

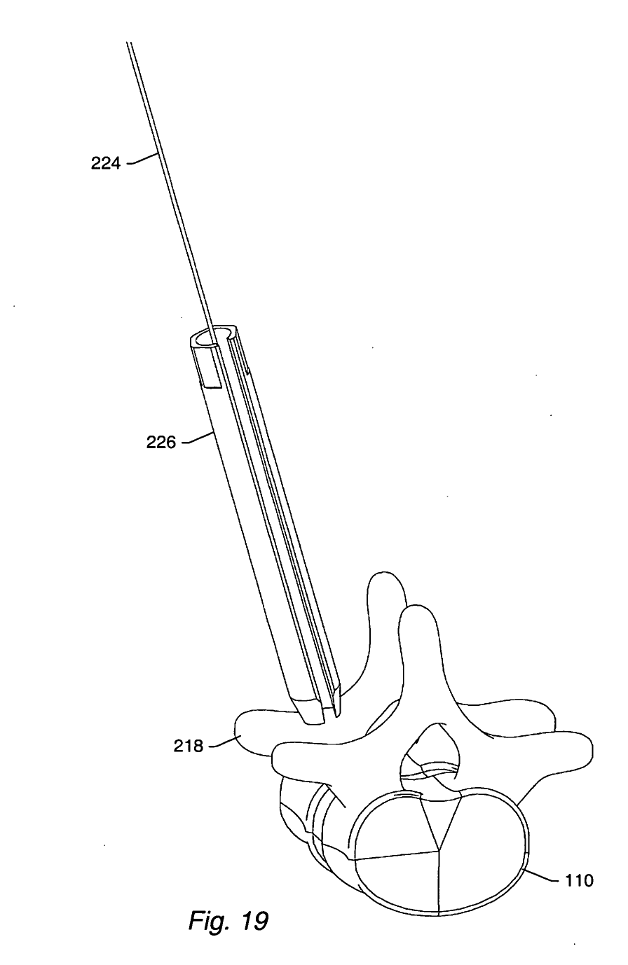

FIG. 19 depicts a perspective view of c-shaped dilator 226 positioned proximate pedicle 218. Rigid member 224 may be positioned in c-shaped dilator 226. The channel down the side of c-shaped dilator 226 may provide access to an adjacent vertebrae for the establishment of a spinal stabilization system.

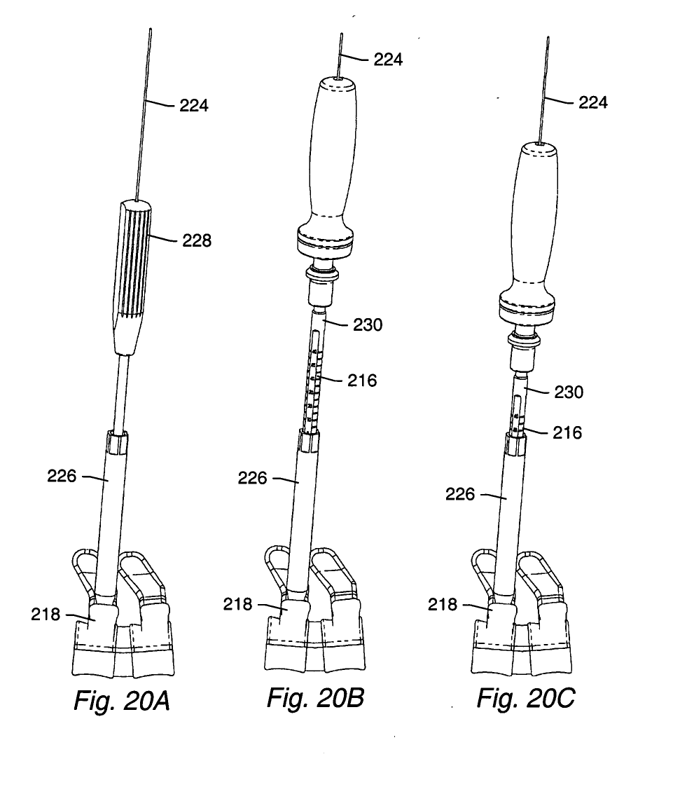

After a c-shaped dilator is positioned adjacent to a pedicle, the pedicle may be prepared to receive a bone fastener. A bone awl may be used to form an opening in the pedicle. FIG. 20A depicts rigid wire 224 positioned through an inner passage of bone awl 228. In some embodiments, a small dilator may be moved down the rigid wire so that a tip of the small dilator is positioned on the top of the bone awl. A mallet or striking device may be used to hit the small dilator so that the bone awl breaches the cortical bone of the pedicle. In some embodiments, the rigid member may be temporarily removed during use of bone awl 228. An outer diameter of a portion of bone awl may substantially correspond to an inner diameter of a c-shaped dilator 226 so that an opening formed by the bone awl is in a desired location. In some embodiments, bone awl 228 may have a variable outer diameter. A small diameter section may include cutting flutes and a cutting surface. A large diameter section may limit insertion depth of the instrument into the bone.

After forming an opening in a pedicle, walls of the pedicle defining the opening may be threaded. FIG. 20B depicts a bone tap positioned in dilator 226. Bone tap 230 may include indicia 216. When bone tap 230 contacts pedicle 218, a first measurement may be taken from indicia 216 relative to top of dilator 226. Bone tap 230 may be advanced into pedicle 218 while monitoring a depth of the bone tap in the bone using a fluoroscope. After bone tap 230 has been advanced into pedicle 218 a desired distance, a second measurement may be taken from bone tap 230 using indicia 216 relative to the top of dilator 226. FIG. 20C depicts bone tap 230 after the bone tap has been driven into pedicle 218. The difference between the two depth measurements may be used to determine a length of a threaded member to be positioned in pedicle 218. After an opening in pedicle 218 has been tapped, bone tap 230 may be removed from dilator 226. In some embodiments, a handle may be removably coupled to the bone tap. In some embodiments, a handle may be an non-removable part of the bone tap.

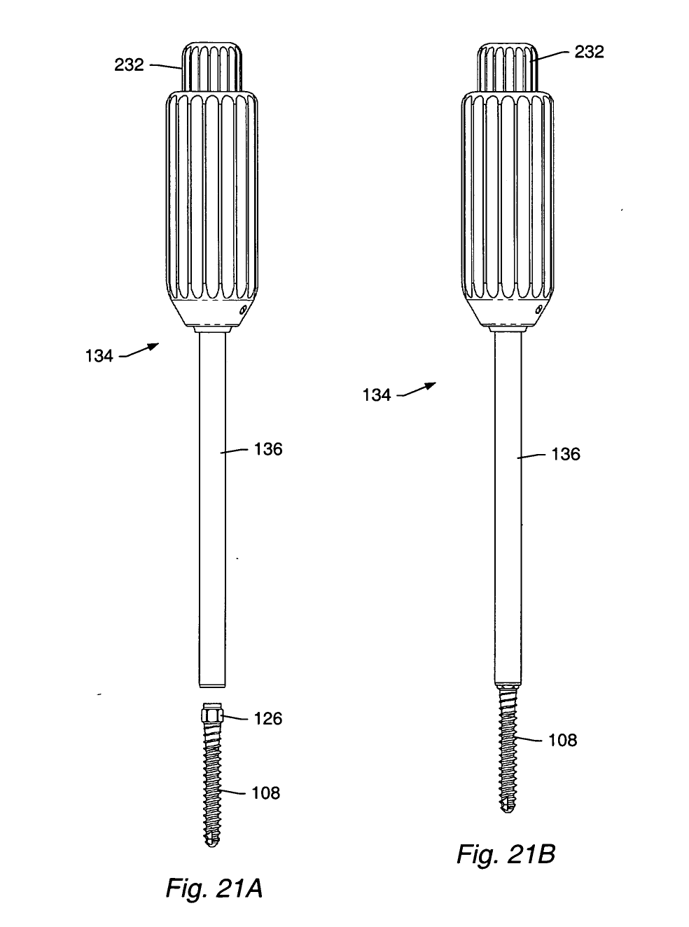

FIG. 21A and FIG. 21B depict embodiments of a driver that may be used to insert a threaded member into a pedicle. Threaded member 108 may be coupled to driver 134. Driver 134 may include an inner shaft and outer shaft 136. The inner shaft may engage an inner surface of threaded member 108. As shown in FIG. 6, an inner surface of threaded member 108 may include threading. A portion of inner surface threading of threaded member 108 may engage the inner shaft of driver 134. Outer shaft 136 may engage tool section 126 of threaded member 108. Driver 134 may include a passage through the driver. The driver passage may be aligned with a passage through threaded member 108 (as shown in FIG. 6). Handle portion 232 of driver 134 may be used to release threaded member 108 after the threaded member is inserted into bone.

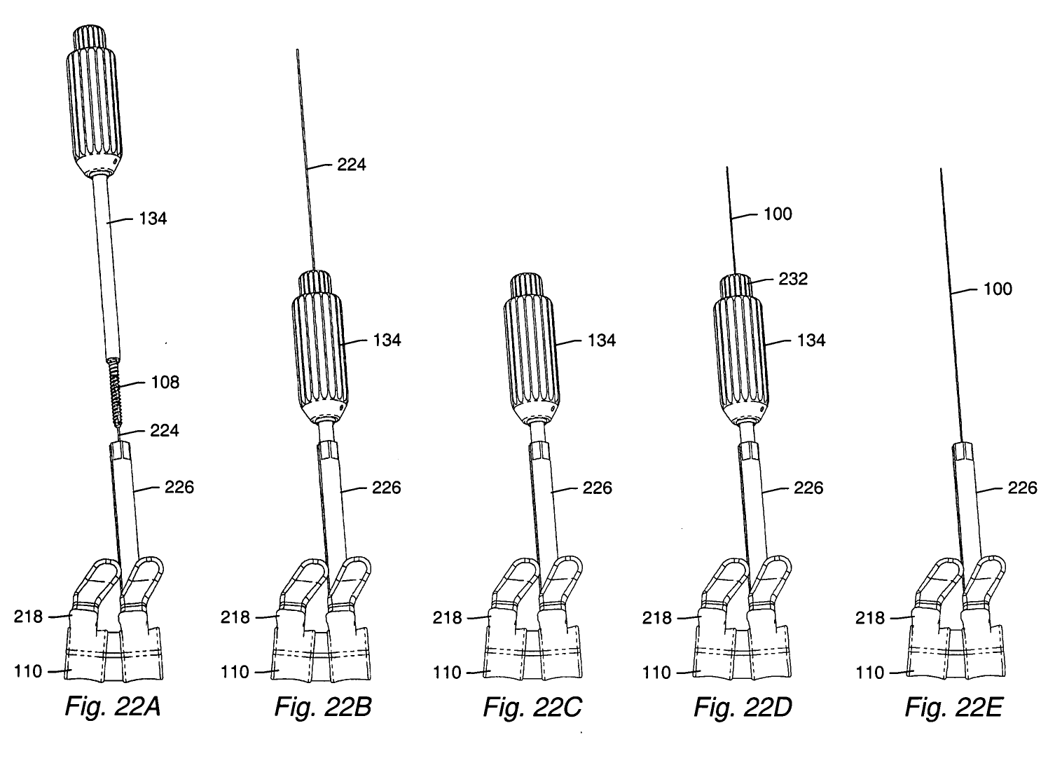

FIG. 22A depicts rigid member 224 partially inserted in driver 134. Driver 134 and threaded member 108 may be advanced along rigid member 224 and into dilator 226 to a position proximate the opening formed in pedicle 218. Driver 134 may be rotated to insert the threaded member into the pedicle. FIG. 22B depicts driver 134 after insertion of the threaded member into the pedicle. After the threaded member is positioned in bone, rigid member 224 may be removed from the pedicle. FIG. 22C depicts driver 134 after the rigid member has been removed. In some embodiments, a flexible member may be inserted through driver 134. The flexible member may be coupled to the threaded member. FIG. 22D depicts flexible member 100 inserted into a passage through driver 134. FIG. 6 depicts a cross-sectional view of flexible member 100 coupled to threaded member 108. In some embodiments, flexible member 100 may engage a portion of the threaded member to couple to the threaded member. After flexible member 100 is positioned in the threaded member, handle portion 232 of driver 134 may be used to release the threaded member from the driver. The driver may be removed from the dilator. FIG. 22E depicts dilator 226 and flexible member 100 after removal of the driver.

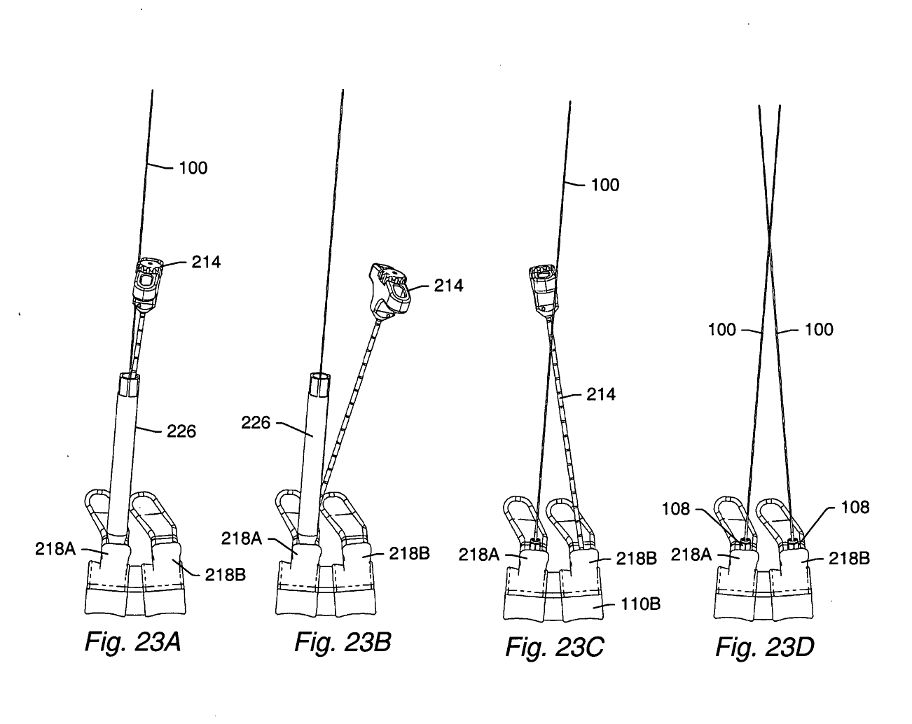

After insertion of a flexible member in a threaded member, a separating member may be positioned in a dilator. FIG. 23A depicts separating member 214 positioned in dilator 226 proximate pedicle 218A. If needed, dilator 226 may be rotated so that a channel in the dilator faces pedicle 218B. In some embodiments, a handle portion of separating member 214 extending above a surface of the body may be positioned over pedicle 218B. FIG. 23B depicts handle of separating member 214 positioned over pedicle 218B. Separating member 214 may be moved through the soft tissue from pedicle 218A to pedicle 218B to separate the soft tissue in a plane between the pedicles. The tissue plane may be formed so that a bottom portion of the formed tissue plane is longer than an upper portion of the tissue plane (i.e., the tissue plane has a substantially trapezoidal shape). The plane may be traced several times to ensure that a well-defined path is formed between pedicle 218A and pedicle 218B. After the plane is formed, the dilator may be removed. FIG. 23C depicts separating member 214 after removal of the dilator. Separating member 214 may be positioned at pedicle 218B such that the separating member may be driven into the pedicle in preparation for inserting a threaded member into vertebra 110B. A threaded member and a flexible member may be inserted into the second pedicle. FIG. 23D depicts pedicle 218A and pedicle 218B with installed threaded members 108 and flexible members 100.

In some embodiments, a tissue wedge may be used instead of a separating member to form the plane between the first pedicle and the second pedicle. A blade of the tissue wedge may have a diamond-shaped cross section with blunted edges. The blade of the tissue wedge may also include a cutting hook that allows fascia to be severed.

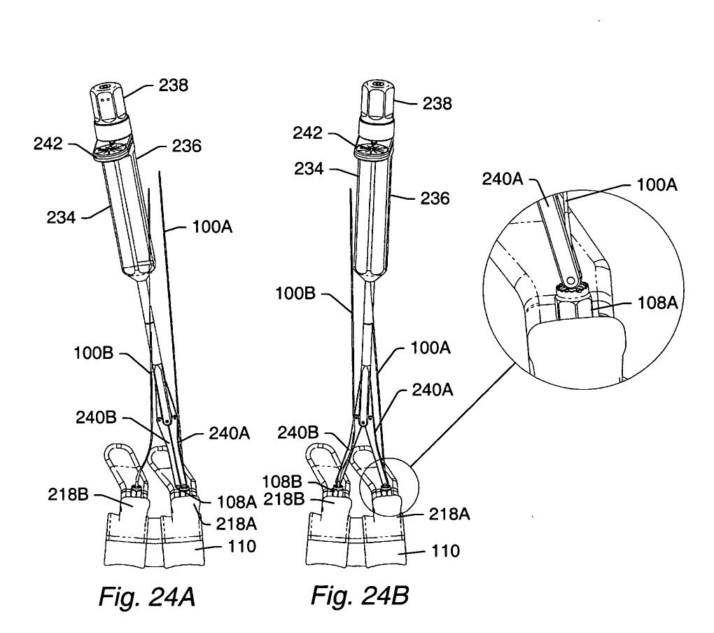

After threaded members and flexible members are installed in pedicles, a length of a coupling mechanism needed to couple the threaded members together may be determined. An estimator tool may be used to determine a distance between threaded members. FIG. 24A and FIG. 24B depict an embodiment of estimator tool 234 during use. Estimator tool 234 may include handle 236; knob 238; measuring arms 240A, 240B; and gauge 242. A user may grip handle 236 when rotating knob 238. Rotating knob 238 may cause measuring arms 240A, 240B to separate from each other or move towards each other depending on the direction that the knob is rotated. When measuring arms 240A, 240B move, an indicator in gauge 242 may indicate an amount of displacement of the ends of the measuring arms relative to each other. In some embodiments, gauge 242 may include two indicators. The first indicator may indicate the current displacement of the arms relative to each other. The second indicator may indicate the maximum displacement that has occurred between the arms. The second indicator may be coupled to a mechanism that allows the second indicator to be reset after use.

Knob 238 of estimator tool 234 may be rotated so that measuring arms 240A, 240B are proximate each other. Flexible member 100A may be passed through an opening in measuring arm 240A. Measuring arm 240A may be guided down flexible member 100A to place an end of the measuring arm in a head of threaded member 108A. Knob 238 may be rotated so that a separation distance between measuring arms 240A, 240B increases. Second measuring arm 240B may follow a tissue plane created between pedicles 218A, 218B that are to be coupled together by a spinal stabilization system. Second measuring arm 240B may include a hook or other engager that couples the measuring arm to flexible member 100B extending from threaded member 108B. Flexible member 100B may be used to help guide the end of second measuring arm 240B to the head of threaded member 108B. The end of second measuring arm 240B may be positioned in the head of threaded member 108B. Positions of measuring arms 240A, 240B may be monitored using fluoroscopy. When measuring arms 240A, 240B are positioned in threaded members 108A, 108B, as depicted in FIG. 24B, a distance between the measuring arms may be read from gauge 242. The measured separation distance may be used to determine a size of a coupling mechanism needed to couple threaded members 108A, 108B together.

In some embodiments, an estimator tool may not include a gauge. Arms of the estimator tool may be coupled to flexible members. The arms may be moved down the flexible members so that a first arm contacts a first threaded member. The estimator tool may be activated so that the arms separate. The second arm may be positioned so that the second arm contacts a second threaded member. The estimator tool may be removed from the patient. During removal, the arms may be compressed. The arms may spring back to the separation distance between the threaded members when fully removed from the patient. A scale (e.g., a scale printed on an instrumentation kit tray) may be used to find a value for the separation distance between the threaded members.

A separation distance between threaded members provided by an estimator tool may be used to determine a size of an elongated member for a spinal stabilization system. Some extra length may be added to the length determined by the estimator tool to account for bending of the elongated member. In some embodiments, the extra length may be equal to or less than 1 cm. In some embodiments, the extra length may be greater than 1 cm.

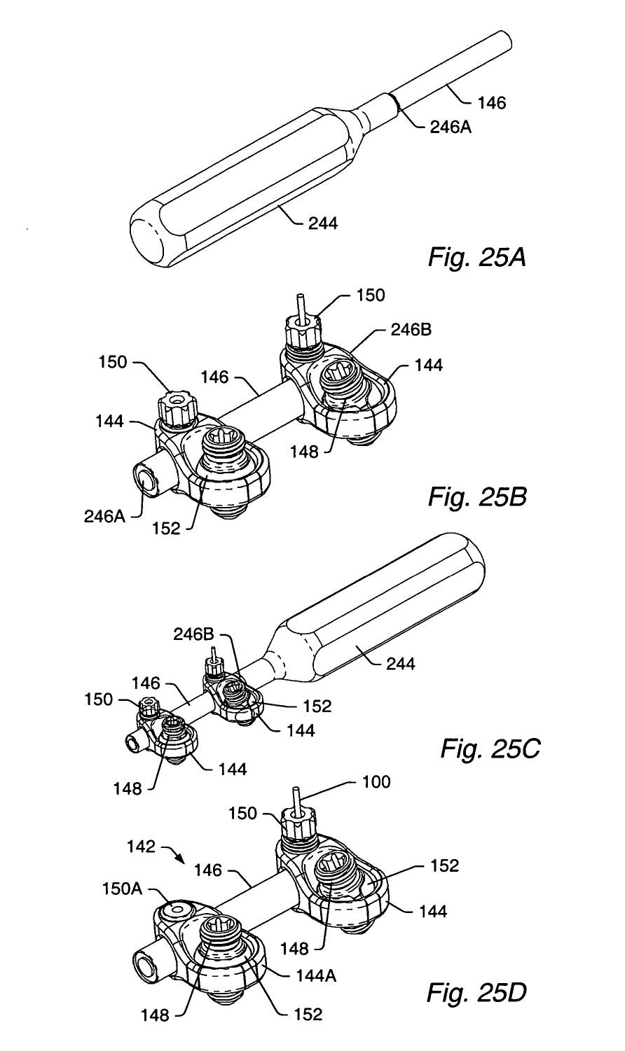

After a desired length for an elongated member is determined, an elongated member of the proper size may be cut. In some embodiments, an end of an elongated member may be flared to inhibit removal of a connector placed on the elongated member. FIG. 25A depicts flare tool 244 that may be used to flare end 246A of elongated member 146.

Connectors may be placed on an elongated member. FIG. 25B depicts elongated member 146 with two connectors 144 placed on the elongated member. End 246A of elongated member 146 may be flared before or after placement of connector 144 on elongated member 146. Flared end 246A may inhibit removal of connectors from elongated member 146. When two connectors 144 are positioned on elongated member 146, second end 246B of the elongated member may be flared to inhibit removal of the connector from the second end of the elongated member. FIG. 25C depicts flare tool 244 positioned to flare end 246B of elongated member 146.

In some embodiments, a position of a first connector on an elongated member may be set by shearing off a head of a setscrew. FIG. 25D depicts a pre-assembled coupling mechanism 142 prior to insertion into the body. The head of setscrew 150A of connector 144A has been sheared off to set the position of the connector relative to elongated member 146. In some embodiments flexible members 100 coupled to setscrews 150 may be positioned in a patient without the position of one of the connectors being fixed relative to the elongated member by shearing off a head of a setscrew.

In some embodiments, such as in the embodiment depicted in FIG. 25D, coupling mechanism 142 may include locking mechanism 148 positioned in ring 152. In other embodiments, a locking mechanism may be coupled to the coupling mechanism during installation of a spinal stabilization system. After insertion and positioning of a coupling mechanism without locking mechanisms against threaded members, a locking mechanism attached to a driver may be moved down a flexible member to the threaded member. The driver may be used to couple threading of the locking mechanism to internal threading of the threaded member.

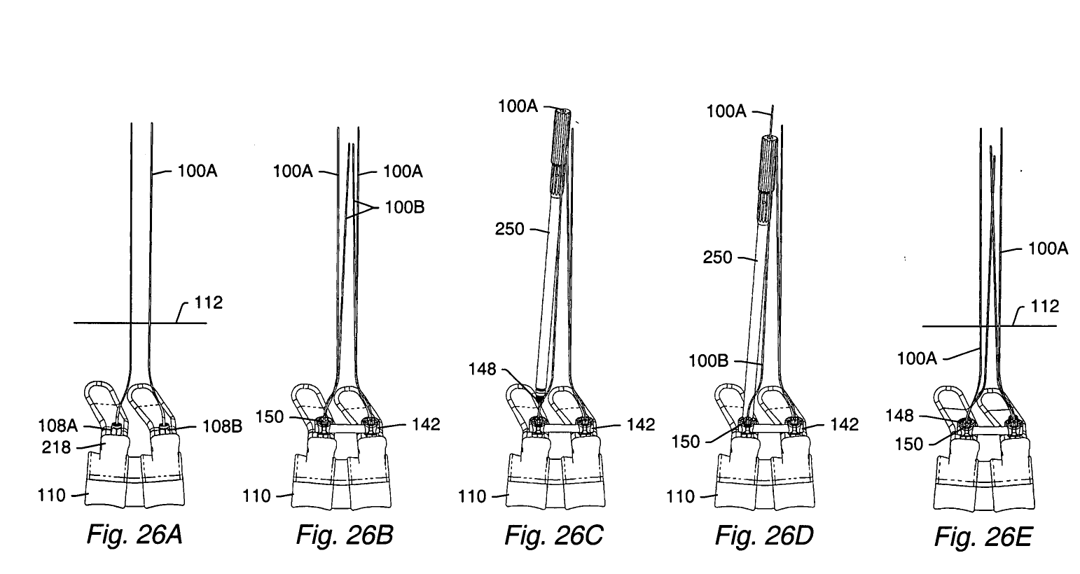

FIGS. 26A-26E depict portions of an installation procedure for an embodiment of a spinal stabilization system. FIG. 26A depicts threaded members 108A, 108B positioned in vertebrae 110. FIG. 26B depicts coupling mechanism 142 positioned against the threaded members. Flexible members 100A may be positioned through rings in coupling mechanism 142. Coupling mechanism 142 may be guided down flexible members 100A to position the rings against the threaded members. Initially, flexible members 100A may be drawn near to each other, and coupling mechanism 142 may be oriented substantially vertically relative to the patient. The substantially vertical orientation may facilitate insertion of coupling mechanism 142 into a small incision at the skin surface. Once past the skin incision, coupling mechanism 142 may be rotated in the tissue plane formed between the threaded members. Coupling mechanism 142 may be guided down flexible members 100A until rings in the coupling mechanism are seated against the threaded members.

Flexible members 100B extend from setscrews 150. In some embodiments, flexible members 100B may be a different color, formed of a different material, be of a different length, or have some other characteristic that distinguishes flexible members 100B from flexible members 100A.

FIG. 26C depicts locking mechanism 148 during insertion. Flexible member 100A is positioned through locking mechanism 148 and a passage in driver 250. Locking mechanism 148 is coupled to driver 250. Locking mechanism 148 may be moved down flexible member 100A to a threaded member. FIG. 26D depicts driver 250 positioned so that the locking mechanism passes through a ring in coupling mechanism 142. Driver 250 is positioned so that the locking mechanism may be secured to the threaded member. Driver 250 may be rotated to secure the locking mechanism to the threaded member. Driver 250 may be removed from the locking mechanism. In some embodiments, driver 250 may be used to shear off a tool portion of the locking mechanism. Driver 250 may retain the sheared-off tool portion of the locking mechanism when the driver is removed from the flexible member. Flexible member 100A may be removed from the threaded member after the tool portion of the locking mechanism is sheared off. FIG. 26E depicts locking mechanism 148 after the tool portion has been sheared off, but before removal of flexible member 100A. The driver may be coupled to a second locking mechanism, and the locking mechanism may be coupled to a second threaded member using flexible member 100A that extends from the second threaded member.

In some embodiments, interbody work may be performed after locking mechanisms couple the connectors to threaded members. The interbody work may include, but is not limited to, installing a fusion device such as a posterior lumbar interbody fusion device, installing a fusion cage, and/or installing a bone graft between the vertebrae.

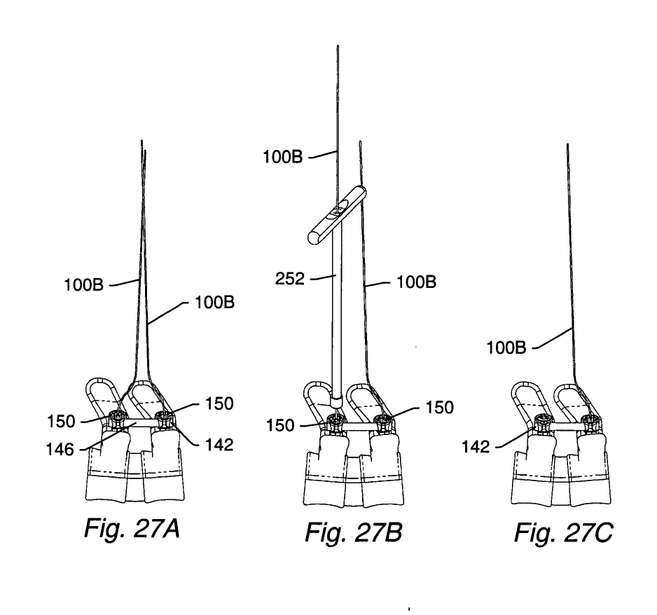

FIG. 27A depicts coupling mechanism 142 with flexible members 100B extending from setscrews 150. After coupling mechanism 142 is securely coupled to threaded members, the position of elongated member 146 relative to connectors 144 may be secured. FIG. 27B depicts driver 252 as the driver is being moved down flexible member 100B towards setscrew 150. Flexible member 100B may be positioned through a passage in driver 252. Flexible member 100B may guide a head of driver 252 to a shear-off portion of setscrew 150. Driver 252 may be coupled to setscrew 150, and the driver may be rotated to break off the shear-off portion of the setscrew. The shear-off portion and flexible member 100B may remain coupled together. The driver, the shear-off portion, and the flexible member may be removed from the patient. FIG. 27C depicts coupling mechanism 142 after a first flexible member has been removed. The driver may be guided down the remaining flexible member 100B. The driver may be used to break off the shear-off portion of the remaining setscrew so that the flexible member can be removed from the coupling mechanism to complete formation of the spinal stabilization system.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A system for stabilizing a spine, comprising:
a first threaded member configured to couple to a first bone during use;
a second threaded member configured to couple to a second bone during use;
a first flexible member configured to couple to the first threaded member during use;
a second flexible member configured to couple to the second threaded member during use; and
wherein the first flexible member and the second flexible member are guides for positioning a coupling mechanism at a desired position relative to the first threaded member and the second threaded member.

2. The system of claim 1, further comprising the coupling mechanism, wherein the coupling mechanism is configured to couple the first threaded member to the second threaded member during use.

3. The system of claim 1, further comprising the coupling mechanism, wherein the coupling mechanism is positionable using the first flexible member and the second flexible member during use, and wherein the coupling mechanism is configured to couple the first threaded member to the second threaded member during use.

4. The system of claim 1, further comprising the coupling mechanism, wherein the coupling mechanism comprises:
a first ring configured to engage a portion of the first threaded member during use; and
a second ring configured to engage a portion of the second threaded member during use.

5. The system of claim 1, further comprising the coupling mechanism, wherein the coupling mechanism comprises a ring configured to engage a portion of the first threaded member or the second threaded member during use.

6. The system of claim 1, further comprising the coupling mechanism, wherein the coupling mechanism comprises:
a first ring comprising protrusions configured to engage protrusions on a head of the first threaded member during use; and
a second ring comprising protrusions configured to engage protrusions on a head of the second threaded member during use.

7. The system of claim 1, further comprising the coupling mechanism, wherein the coupling mechanism comprises:
a first connector configured to engage the first threaded member positioned in bone;
a second connector configured to engage the second threaded member positioned in bone; and
an elongated section configured to couple the first connector to the second connector.

8. A system for stabilizing a spine, comprising:
a first threaded member configured to couple to a first vertebra during use;
a second threaded member configured to couple to a second vertebra during use; and
a coupling mechanism comprising:
a first connector configured to engage a portion of the first threaded member during use;
a second connector configured to engage a portion of the second threaded member during use; and
an elongated member configured to couple to the first connector and the second connector such that the first vertebra is coupled to the second vertebra; and
wherein at least one of the threaded members comprises an inner conduit configured to couple to a flexible member during use.

9. The system of claim 8, further comprising one or more guiding mechanisms configured to position the coupling mechanism proximate the first threaded member and the second threaded member through an opening in soft tissue during use.

10. The system of claim 8, wherein at least one of the connectors comprises a curvate wall to engage a portion of a ring during use.

11. The system of claim 8, wherein the first threaded member comprises a threading, and wherein the threading is configured to engage threading of a flexible member.

12. The system of claim 8, further comprising:
a ring configured to couple at least one of the threaded members to at least one of the connectors during use; and
wherein at least one of the connectors is configured to frictionally lock the ring.

13. A method of stabilizing vertebrae, comprising:
coupling a first member of a stabilization system to a first vertebra; and
moving a separating member from the first vertebra to a second vertebra through soft tissue to separate the soft tissue substantially on a plane between the first vertebra and the second vertebra without severing the soft tissue.

14. The method of claim 13, wherein the separating member comprises a needle.

15. The method of claim 13, further comprising coupling a second member of the spinal stabilization system to the second vertebra, and providing a coupling mechanism to connect the first member to the second member.

16. The method of claim 15, further comprising forming an opening through soft tissue to allow access to the first vertebra, wherein the opening is less than about 4 cm in length at a surface of the skin.

17. The method of claim 15, further comprising coupling a first flexible member to the first member, coupling a second flexible member to the second member, and guiding the coupling mechanism toward the first member and the second member using the first flexible member and the second flexible member.

18. The method of claim 15, further comprising adjusting a length between connectors of the coupling mechanism.

19. The method of claim 15, further comprising adjusting a length between connectors of the coupling mechanism, and setting the length between the connectors by shearing off a head of a setscrew.

20. The method of claim 15, further comprising positioning the coupling mechanism using a first guide coupled to the first member and a second guide coupled to the second member, and removing the first guide from the first member and the second guide from the second member.

21. A flexible member for a spinal stabilization system, comprising:
a first section comprising a first stiffness;
a second section comprising a second stiffness; and
wherein the stiffness of the second section is greater than the stiffness of the first section.

22. The member of claim 21, wherein the flexible member is configured to engage a threaded member during use.

23. The member of claim 21, wherein stiffness between the first and second sections gradually increases from about the first stiffness to about the second stiffness.

24. The member of claim 23, wherein the flexible member is configured to maintain alignment of the flexible member along a centerline of the threaded member within about 2 cm or less of a head of the threaded member during use.

25. The member of claim 23, wherein the flexible member is configured to maintain alignment of the flexible member along a centerline of the threaded member within about 1.3 cm or less of a head of the threaded member during use.

26. The member of claim 23, wherein the flexible member is configured to maintain alignment of the flexible member along a centerline of the threaded member within about 1 cm or less of a head of the threaded member during use.

27. The member of claim 21, wherein the first section has a first thickness, wherein the second section comprises a second thickness, and wherein the second thickness is greater than about the first thickness.

28. The member of claim 21, wherein the flexible member comprises a cable.

29. The member of claim 21, wherein the flexible member comprises a wire.

30. The member of claim 21, wherein the flexible member is configured to couple to a threaded member to guide components of a spinal stabilization system to a surgical site during use.

31. The member of claim 21, further comprising a threaded member, wherein the flexible member is configured to couple to the threaded member to guide components of the spinal stabilization system to a surgical site during use.

32. A system for stabilizing a spine, comprising:
a first threaded member configured to couple to a first portion of bone during use;
a second threaded member configured to couple to a second portion of bone during use;
a first flexible member configured to couple to the first threaded member; and
a second flexible member configured to couple to the second threaded member during use.

33. The system of claim 32, further comprising a coupling mechanism configured to couple the first threaded member to the second threaded member during use.

34. The system of claim 32, further comprising a coupling mechanism positionable using the first flexible member and the second flexible member during use, and wherein the coupling mechanism is configured to couple the first threaded member to the second threaded member during use.

35. The system of claim 32, further comprising a coupling mechanism comprising:
a first ring configured to engage a portion of the first threaded member during use; and
a second ring configured to engage a portion of the second threaded member during use.

36. The system of claim 32, wherein at least one of the flexible members comprises a cable.

37. The system of claim 32, wherein at least one of the flexible members comprises a variable thickness cable.

38. The system of claim 32, wherein at least one of the flexible members comprises a stopping mechanism.

39. The system of claim 32, further comprising a coupling mechanism comprising:
a first connector configured to engage the first threaded member positioned in bone;
a second connector configured to engage the second threaded member positioned in bone; and
an elongated section configured to couple the first connecting section to the second connecting section.

40. The system of claim 32, further comprising a coupling mechanism comprising at least one connector configured to engage a threaded member during use.

41. The system of claim 32, wherein the first flexible member is positionable through the first threaded member opening in a coupling mechanism during use.

42. The system of claim 32, wherein the first flexible member is positionable through the first threaded member opening in a coupling mechanism during use, and wherein the second flexible member is positionable through a second threaded member opening in the coupling mechanism during use.

43. A bone stabilization system, comprising:
a threaded member comprising one or more protrusions on a head of the threaded member;
a ring configured to engage protrusions on the head of the threaded member during use; and
a coupling mechanism configured to engage the threaded member during use comprising:
an opening through a connector configured to engage the threaded member during use;
a locking mechanism configured to couple the threaded member to the ring during use; and
wherein the system is configured such that interaction of protrusions on the head of the threaded member and the ring inhibits rotation of the threaded member in the bone during use.

44. The system of claim 43, wherein the one or more protrusions comprise one or more teeth.

45. The system of claim 43, wherein an inner surface of the locking mechanism is configured to engage a first tool as the locking mechanism is advanced into the threaded member during use.

46. The system of claim 43, wherein an inner surface of the locking mechanism is configured to engage a first tool as the locking mechanism is advanced into the threaded member during use, and wherein an outer surface of the locking mechanism is configured to be engaged by a second tool as the locking mechanism is tightened during use.

47. The system of claim 43, wherein an inner surface of the locking mechanism is configured to engage a first tool as the locking mechanism is advanced into the threaded member during use, wherein an outer surface of the locking mechanism is configured to be engaged by a second tool as the locking mechanism is tightened during use, and wherein a portion of the locking mechanism is configured to be removed by the second tool during use.

48. The system of claim 43, wherein the coupling mechanism comprises a plate.

49. The system of claim 43, wherein the coupling mechanism comprises an elongated member.

50. The system of claim 43, wherein the coupling mechanism is adjustable.

51. A ring configured to couple a threaded member to a coupling mechanism during use, comprising:
a first surface configured to engage a wall of the coupling mechanism during use;
a second surface configured to engage a locking mechanism during use; and
a third surface comprising one or more teeth configured to engage a portion of the threaded member during use such that rotational movement of the threaded member in bone during use is inhibited.

52. The ring of claim 51, wherein the first surface comprises titanium.

53. The ring of claim 51, wherein a portion of the wall of the coupling mechanism cuts into the first surface of the ring during use.

54. The ring of claim 51, wherein the ring comprises one or more slots.

55. The ring of claim 51, wherein the ring is substantially “C” shaped.

56. The ring of claim 51, wherein the ring comprises a circular structure with a gap in the circular structure.

57. The ring of claim 51, wherein the second surface is substantially harder than the first surface.

58. The ring of claim 51, wherein the ring inhibits backout of the threaded member from the coupling mechanism during use.

59. The ring of claim 51, wherein the ring is positionable in the threaded member opening between the coupling mechanism and a locking mechanism.

60. The ring of claim 51, wherein the ring comprises titanium.

61. The ring of claim 51, wherein the ring further comprises a gap to allow the ring to expand and contract.

62. The ring of claim 51, wherein the ring comprises a ledge configured to engage a portion of a locking mechanism during use.

63. The ring of claim 51, wherein a wall of the connector is configured to frictionally lock with the ring during use.

64. The ring of claim 51, wherein a wall of the connector is roughened.

65. A method of stabilizing a spine, comprising:
coupling a first threaded member to a first vertebra;
establishing a plane of separated tissue between the first vertebra and a second vertebra; and
coupling a second threaded member to the second vertebra.

66. A method of stabilizing a spine, comprising:
accessing a first portion of the spine through an opening in soft tissue;
coupling a first threaded member of a spinal stabilization system to the first portion of the spine;
establishing a plane of separated tissue between the first portion of the spine and a second portion of the spine;
accessing the second portion of the spine through the plane of separated tissue;
coupling a second threaded member of the spinal stabilization system to the second portion of the spine;
providing a coupling mechanism of the spinal stabilization system to the plane of separated tissue;
coupling a first section of the coupling mechanism to the first portion of the spine; and
coupling a second section of the coupling mechanism to the second portion of the spine.

67. The method of claim 66, further comprising positioning a third member of the spinal stabilization system proximate the first member and the second member.

68. The method of claim 66, further comprising coupling a third threaded member of the spinal stabilization system to the first threaded member and the second threaded members.

69. The method of claim 66, further comprising positioning the coupling mechanism proximate the first vertebra and the second vertebra using a first guide mechanism and a second guide mechanism.

70. A method of stabilizing a spine, comprising:
accessing a portion of the spine through an opening in soft tissue;
coupling a flexible member to a first vertebra in the portion of the spine;
coupling a second flexible member to a second vertebra in the portion of the spine;
positioning a coupling mechanism proximate the first vertebra and the second vertebra using a first guide mechanism and a second guide mechanism;
coupling a first section of the coupling mechanism to the first vertebra; and
coupling a second section of the coupling mechanism to the second vertebra.
